(19) United States
(12) Patent Application Publication
(10) Pub. No.: US 2007/0098649 A1
(43) Pub. Date: May 3, 2007

(54) Method and composition for controlling oral pathogens

(75) Inventors: Christine D. Wu, Chicago, IL (US); A. Douglas Kiinghorn, Columbus, OH (US); Sara Kate Roberts, Wellington (NZ)

Correspondence Address: GREENLEE WINNER AND SULLIVAN P C, 4875 PEARL EAST CIRCLE, SUITE 200, BOULDER, CO, 80301, US

(73) Assignee: THE BOARD OF TRUSTEES OF THE UNIVERSITY OF ILLINOIS, URBANA (IL)

(21) Appl. No.: 10/551,639

(22) PCT Filed: Mar. 22, 2004
(86) PCT No.: PCT/US04/08616

§ 371 Date: Dec. 14, 2006

(51) Int. Cl. A61K 8/49; A61K 31/65

(52) U.S. Cl. 424/49; 514/154; 514/283

(57) ABSTRACT

A composition and method of controlling oral and other human pathogens is disclosed. The composition and method utilize an antimicrobial or antibiotic and a berberine as active agents to treat mammals, including humans.

### STATEMENT OF GOVERNMENTAL INTEREST

The subject matter of this application has been supported by research Grant No. NIDCR/NIH DE13990-02, from the National Institute for Dental and Craniofacial Research. The government may have an interest in this invention.

### FIELD OF THE INVENTION

The present invention relates to the control of oral and other human pathogens, and to the treatment of diseases and conditions that directly or indirectly result from such pathogens, by administering (a) berberine and (b) an antimicrobial or an antibiotic. More particularly, the present invention relates to a method of potentiating the effects of an antimicrobial or an antibiotic in a mammal by administration of therapeutically effective amounts of both berberine and an antimicrobial or an antibiotic. The composition and method of the present invention permit a reduction in the antimicrobial or antibiotic dose and in the berberine dose while providing a desired therapeutic effect, thus reducing adverse side effects.

### BACKGROUND OF THE INVENTION

Dental plaque frequently is associated with oral diseases, including dental caries and periodontal disease (i.e., gum disease). Plaque control can be achieved by mechanical (e.g., brushing and flossing) or chemical means. For example, antimicrobial agents have been incorporated into oral hygiene products, such as toothpastes and mouth rinses, to control plaque and gingivitis/periodontal disease.

At present, the antimicrobial chlorhexidine (CHX) is the “gold standard” of plaque control agents and is included in several oral hygiene products. However, CHX has many adverse side effects, including an undesirable taste, tooth discoloration, and increased calculus (i.e., tartar) formation.

Tetracyclines are conventional antibiotics, and routinely are used in the treatment of periodontal disease because of their unique ability to accumulate in the periodontal pocket area. However, like other conventional antibiotics, many bacteria, especially important human pathogens, have developed a resistance to antibiotics. Tetracycline antibiotics also have many adverse side effects, including gastrointestinal discomfort, nausea, vomiting, diarrhea, and permanent tooth discoloration in children up to eight years old.

Berberine is a plant alkaloid having a long history of medicinal use in both Ayurvedic and Chinese medicine. Berberine is present in the roots, rhizomes, and stem bark of Hydrastis canadensis (goldenseal), Coptis chinensis (coptis or goldenthread), Berberis aguifolium (Oregon grape), Berberis vulgaris (barberry), and Berberis aristata (tree tumeric). Berberine is 5,6-dihydro-9,10-dimethoxybenzo[g]-1,3-benzodioxolo[5,6-a]quinolizinium, and as used herein, the term “berberine” includes free berberine and salts thereof.

Goldenseal is a major phytomedicine sold in the United States and is one of the top five herbal supplements in the worldwide market. Goldenseal is used in various dietary supplements for the purpose of enhancing the immune response of the body. Goldenseal-containing oral hygiene products also are available over the counter, but limited information exists with respect to the efficacy of goldenseal in oral health applications.

Berberine extracts and decoctions demonstrate significant antimicrobial activity against a variety of organisms, including bacteria, viruses, fungi, protosoans, helminthes, and chlamydia. Currently, the predominant clinical uses of berberine include treatment of bacterial diarrhea, intestinal parasite infections, and ocular trachoma infections.

As previously stated, antimicrobials and antibiotics exhibit several adverse side effects, including tooth discoloration, bad taste, and nausea, but they are routinely used in the treatment of periodontal disease. In accordance with the present invention, a combination treatment utilizing berberine and a conventional antimicrobial or antibiotic agent allows for decreased concentrations of both agents, which reduces the occurrence or severity of adverse side effects. Such a finding is an important advance in the art. The adverse side effects attributed to antibiotics limit the usefulness of antimicrobials and antibiotics, like tetracyclines, and antimicrobials, like CHX, in the treatment and control of diseases and conditions of the mouth, teeth, and gums. The present invention, therefore, is directed to the discovery that an antimicrobial or antibiotic and berberine, when administered in combination, perform synergistically. Therefore, the amount of antimicrobial or antibiotic and berberine used to control the oral pathogens can be reduced, with a concomitant reduction or elimination of adverse side effects. The combined use of an antimicrobial or antibiotic and a berberine also can be used to synergistically control nonoral human-pathogens.

Additional background information can be found in Appendix A, which constitutes a portion of this disclosure.

### SUMMARY OF THE INVENTION

In a programmed research effort, it was found that administration of berberine potentiates the antimicrobial effects of an antimicrobial or antibiotic in the treatment of oral and other human pathogens. Administration of an antimicrobial or antibiotic (hereafter “antibiotic”) in combination with berberine synergistically potentiates the antimicrobial effect of an antibiotic, and, therefore, lowers the dose of both the antibiotic and berberine required to provide a desired antimicrobial effect. The reduced amount of antibiotic and berberine required to provide a desired antimicrobial effect reduces the severity of various adverse side effects associated with antibiotic and berberine treatment.

Accordingly, one aspect of the present invention is to provide a composition comprising (a) an antibiotic, e.g., CHX, tetracycline, or doxycycline, and (b) berberine, or a salt, derivative, or prodrug thereof, for use in controlling oral and other human pathogens.

The present invention, therefore, provides a composition and method for improved control of oral pathogens, and treatment of diseases and conditions caused by oral pathogens, like gingivitis and dental caries. In particular, the present invention is directed to compositions containing berberine and an antibiotic, and to methods of using the composition to prevent and/or treat diseases and conditions of the mouth, teeth, and gums. More particularly, the present invention is directed to compositions containing berberine and CHX or a tetracycline, and to use of the compositions in methods of treating diseases and conditions of the mouth, teeth, and gums.

Another aspect of the present invention is to provide a method and composition for controlling oral pathogens, and for treating conditions and diseases caused by such pathogens, while reducing the adverse side effects associated with berberine and antibiotic treatment.

Yet another aspect of the present invention is to provide a method and composition for controlling nonoral human pathogens, and treating conditions and diseases directly or indirectly caused by such pathogens.

Still another aspect of the present invention is to provide a method and composition for controlling oral and nonoral human pathogens, including antibiotic-resistant pathogens.

Another aspect of the present invention is to provide an article of manufacture for human use comprising (a) a package insert, (b) a container, and either (c1) a packaged composition comprising an antibiotic and berberine or (c2) a packaged composition comprising an antibiotic and a packaged composition comprising berberine.

These and other aspects of the present invention will become apparent from the following detailed description of the preferred embodiments of the invention.

### DETAILED DESCRIPTION OF THE PREFERRED EMBODIMENTS

The present invention also is described in detail in the attached Appendix A, which constitutes a portion of the present disclosure.

In particular, Appendix A discloses test results relating to administration of a berberine and an antibiotic to control oral pathogens. The tests show that administration of berberine synergistically potentiates the antimicrobial effect of an antibiotic, and allows a reduction in the dose of both the antibiotic and berberine, while providing an antimicrobial effect equivalent to using a higher dose of antibiotic or berberine used alone. Such a synergistic coadministration provides an effective treatment, reduces adverse side effects associated with antibiotic or berberine administration, and addresses the problem of bacterial resistance to antibiotics.

The present invention, therefore, provides methods of synergistically potentiating the antimicrobial properties of an antibiotic and berberine in the treatment of oral and other human pathogens. The invention also provides pharmaceutical compositions comprising an antibiotic and berberine.

The present invention also provides articles of manufacture comprising an antibiotic and berberine, packaged separately or together, and an insert having instructions for using these active agents in the control of oral and other human pathogens, and the treatment of diseases and conditions caused by such pathogens.

The methods described herein benefit from, the use of an antibiotic and berberine in the treatment and management of oral and other human pathogens. The antibiotic and berberine can be administered simultaneously or sequentially to achieve the desired synergistic antimicrobial effect.

For the purposes of the description herein, including Appendix A, the term “treatment” includes preventing, controlling, lowering, or eliminating the concentration of oral and other human pathogens. As such, the term “treatment” includes both medical therapeutic and/or prophylactic administration, as appropriate.

The term “container” means any receptacle and optional closure therefor suitable for storing, shipping, dispensing, and/or handling an article of manufacture.

The term “insert” means information accompanying an article of manufacture that provides a description of how to administer the article, along with the safety and efficacy data required to allow the physician, pharmacist, or user to make an informed decision regarding use of the article. The package insert generally is regarded as the “label” for an article.

The term “prodrug” means compounds that transform rapidly in vivo to a compound useful in the invention, for example, by hydrolysis. A thorough discussion of prodrugs is provided in Higuchi et al., Prodrugs as Novel Delivery Systems, Vol. 14, of the A.C.S.D. Symposium Series, and in Roche (ed.) Bioreversible Carriers in Drug Design, American Pharmaceutical-Association and Pergamon Press, 1987.

In accordance with an important feature of the present invention, an antibiotic and a berberine are present in a composition, or are administered in combination; in a weight ratio of antibiotic-to-berberine of about 0.01:1 to about 100:1, and preferably about 0.01:1 to about 10:1. This ratio depends upon the type and identity of antibiotic and berberine being used. The ratio of antibiotic-to-berberine that is administered is dependent upon the particular antibiotic and berberine used, and the origin and severity of the condition being treated. This ratio can be readily determined by a person skilled in the art to achieve the desired control of an oral or other human pathogen.

When berberine is used in combination with an antibiotic, such as CHX or a tetracycline, like doxycycline, a synergistic antimicrobial growth inhibition effect is observed. Such a combination treatment results in a lower minimum inhibitory concentration (MIC) of either active agent.

Antibiotics most commonly are used in dental practice as prophylactic agents for preventative management of endocarditis. In addition, antibiotics are used therapeutically in cases where infections of oral hard and soft tissues, such as teeth and gingival, cannot be controlled by local debridement and can spread to distant organs, and, therefore, require supplemental therapy.

Tetracycline antibiotics have a broad spectrum of activity, can be used by many routes of administration, such as oral administration and sustained delivery systems, and are widely used. Tetracyclines inhibit protein synthesis by combining the small (30S) subunit of the ribosome and inhibiting the binding of aminocyl-tRNA molecules to the ribosomal A site. Antibiotics generally are considered bacteriostatic.

Reports of bacterial resistance to tetracyclines, both in vitro and in vivo, are increasing. However, antibiotics still are useful, and are routinely used in periodontal treatment. Tetracyclines concentrate in periodontal pockets at inhibitory levels 2 to 4 times higher than in the blood, strongly bind to root surfaces, and can be released in active form over extended time periods. Sublethal concentrations of tetracyclines reduce adherence and coaggregation properties of a number of pathogens and also inhibit the collagenolytic enzymes of mammalian cells enabling periodontal ligament regeneration. But, if the pathogen is a resistant to antibiotics, the periodontal disease may not be treated, but rather may be aggravated.

Chlorhexidine (CHX) is a bisbiguanide biocide, and is marketed in the form of different salts. Chlorhexidine gluconate is one of the most common of these salts. CHX-containing compositions have been used as topical disinfectants since the middle 1970s. Because chlorhexidine in the salt form is cationic, it readily binds to negatively charged cell walls. CHX targets the cytoplasmic membrane of bacteria resulting in the loss of structural organization and integrity. Subsequently, coagulation and precipitation of cytoplasmic constituents occurs.

In patients having high caries activity and high counts of Streptococci mutans, CHX can be employed as an adjunct to other preventative measures. In periodontal treatment, chlorhexidine is used for postoperative rinsings and as an adjunct to mechanical debriment. CHX compositions also are used for nonspecific plaque control, as well as against specific bacteria associated with periodontal disease and caries and in subgingival irrigation. CHX usually is administered from rinsing solutions (concentrations, typically about 1-2%), and slow release devices, such as varnishes, fibers, and slab-like sustained delivery devices. CHX also can be incorporated into chewing gum, toothpastes, and toothbrushes, or in an oral rinse or in a disinfectant for dentures in patients with candidial infections. CHX has a low level of toxicity both locally and systemically and shows no permanent retention in the body.

The antibiotic incorporated into a present composition or utilized in a present method is not limited to CHX. Any other antibiotic useful in the treatment of oral and other human pathogens, like the tetracyclines, can be used in the present composition and methods. Examples of useful antibiotics include, but are not limited to, chlorhexidine, chlorhexidine dihydrochloride, chlorhexidine diacetate salt hydrate, chlorhexidine digluconate, alexidine, alexidine dihydrochloride, tetracycline, tetracycline hydrochloride, doxycycline, doxycycline hydrochloride, and mixtures thereof.

The second active agent in a present composition and method is berberine. In humans, berberine markedly improves cardiac performance in patients with heart conditions when administered at 0.02 mg/kg/min (59 nmol/kg/min) for 30 minutes, followed by 0.2 mg/kg/min (0.59 mol/kg/min) for an additional 30 minutes. However, berberine administration to heart failure patients can result in ventricular tachycardia in some subjects. Berberine also has an anesthetic effect when injected s.c., and berberine produced parapsympatholytic and anesthetic effects when applied to the eyes.

Berberine sulfate is absorbed through human skin. Following oral administration, berberine is absorbed slowly, requiring four hours to reach peak concentrations in plasma and another four hours to clear. In rats orally administered tritiated berberine chloride, blood levels of the compound leveled after 4 to 24 hours; peak levels in liver and muscles occurred at 12 hours, while urinary excretion peaked at 12 to 24 hours. After 48 hours, the majority of the administered dose had been excreted in feces. Following intravenous administration to rats, the highest concentrations of berberine were found in the kidneys, with lower concentrations in the liver, lung, and brain. In rabbits, small amounts of berberine were found in the heart, liver, and kidneys 24 hours after administration by gavage.

In mice, the oral LD₅₀ dose of berberine is 329 mg/kg (0.98 mmol/kg), the s.c. LD₅₀ dose is, 18 mg/kg (0.054 mmol/kg), and the i.p. LD dose is greater than 500 mg/kg (>1.49 mmol/kg (>2.31 mmol/kg) orally. In rabbits, the s.c. LD₅₀ dose is 100 mg/kg (0.30 mmol/kg).

When tested as protection from amphetamine toxicity, berberine chloride (5 mg/kg; 0.013 mmol/kg), and berberine sulfate (15 mg/kg; 0.035 mmol/kg), injected i.p. had no significant effect in mice. Berberine sulfate reduced rectal temperature in albino rats injected i.p. with 50 mg/kg (0.12 mmol/kg), and a single intraintestinal injection (10 mg/kg; 0.023 mmol/kg) into the duodenum of rats had no effect on the volume or acidity intravenously at 6 mg/kg (0.014 mmol/kg). Berberine sulfate significantly increased the number of apomorphine-induced vomits in dogs, and reduced the blood pressure in rats, dogs, and cats following intravenous administration of 0.1-6.0 mg/kg (0.00023-0.014 mmol/kg).

A recent publication disclosed that berberine administered in combination with pyrimethamine gave good results in the treatment of patients suffering from chloroquine-resistant malaria. It also has been reported that berberine, isolated from the Chinese medicinal plant, Coptidis rhizoma, has antimicrobial activity against seven periodontal pathogens, and that berberine, therefore, possibly can be used in a clinical treatment of periodontal diseases.

The present invention is directed to the use of berberine, a natural compound found in goldenseal, in combination with an antibiotic, e.g., CHX or a tetracycline, in the control of oral and other human pathogens, and diseases and conditions directly or indirectly caused by such pathogens. The invention is not limited to the use of a particular berberine, but extends to free berberine, all pharmaceutically acceptable salts of berberine, derivatives of berberine, and prodrugs of berberine.

Examples of berberines and berberine derivatives useful in the present invention include, but are not limited to, free berberine, berberine chloride, berberine bisulfate, berberine hemisulfate, a berberine prodrug, a berberine derivative listed in the following Table 1, and mixtures thereof.

TABLE 1

| | Molecular | Molecular | |
|---|---|---|---|
| Name | Formula | Weight | Structure |
| berberine chloride | C₂₀H₁₈ClNO₄ | 371.81 | 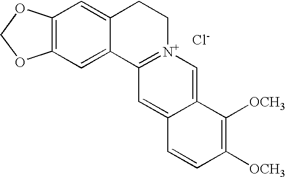 |
| berberrubine | C₁₉H₁₆NO₄ | 322.33 | 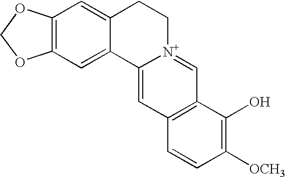 |
| coptisine | C₁₉H₁₄NO₄ | 320.32 | 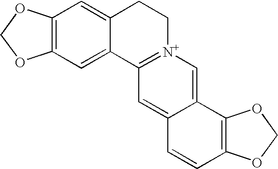 |
| palmatine | C₂₁H₂₂NO₄ | 352.40 | 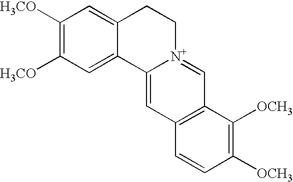 |
| 8-ethyl-12-bromoberberine | C₂₂H₂₁BrNO₄ | 443.31 | 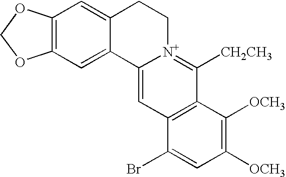 |
| 8-ethylberberine | C₂₂H₂₂NO₄ | 364.41 | 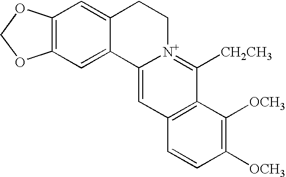 |
| 8-methoxyberberine | C₂₁H₂₀NO₅ | 366.39 | 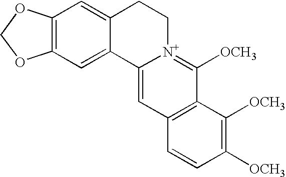 |
| 8-methylberberine | C₂₁H₂₀NO₄ | 350.39 | 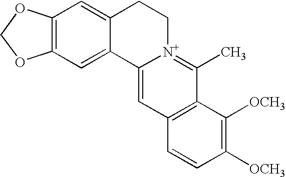 |
| 8-n-butyl-12-bromoberberine | C₂₄H₂₅BrNO₄ | 471.36 | 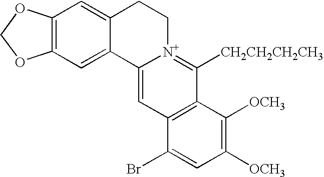 |
| 8-n-butylberberine | C₂₄H₂₆NO₄ | 392.47 | 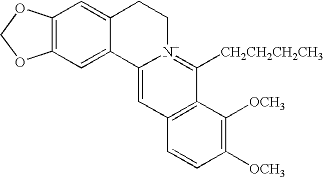 |
| 8-n-hexyl-12-bromoberberine | C₂₆H₂₉BrNO₄ | 499.42 | 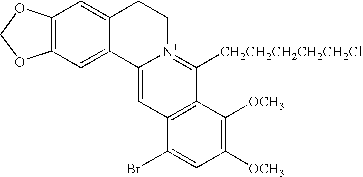 |
| 8-n-propyl-12-bromoberberine | C₂₃H₂₃BrNO₄ | 457.34 | 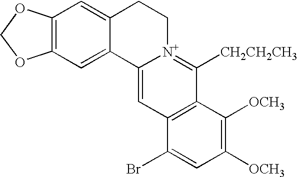 |
| 8-n-propylberberine | C₂₃H₂₄NO₄ | 378.44 | 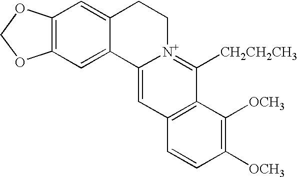 |
| 8-phenyl-12-bromoberberine | C₂₆H₂₁BrNO₄ | 491.35 | 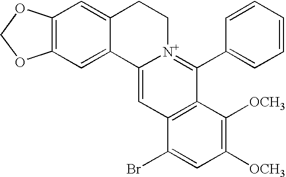 |
| 8-phenylberberine | C₂₆H₂₂NO₄ | 412.46 | 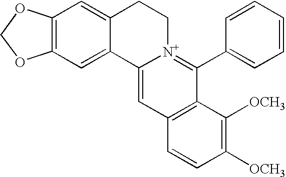 |
| 9-O-acetylberberrubine | C₂₁H₁₈NO₅ | 364.37 | 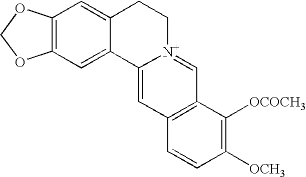 |
| 9-O-benzoylberberrubine | C₂₆H₂₀NO₅ | 426.44 | 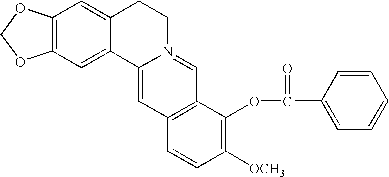 |
| 9-O-ethyl-13- ethylberberrubine | C₂₃H₂₄NO₄ | 378.44 | 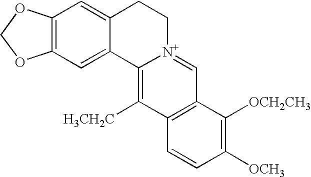 |
| 9-O-ethylberberrubine | C₂₁H₂₀NO₄ | 350.39 | 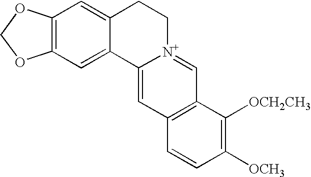 |
| 9-O-lauroylberberrubine | C₃₁H₃₈NO₅ | 504.64 | 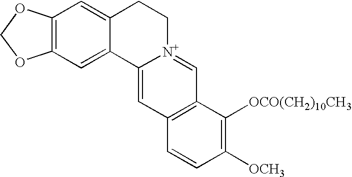 |
| 9-O-valerylberberrubine | C₂₄H₂₄NO₅ | 406.45 | 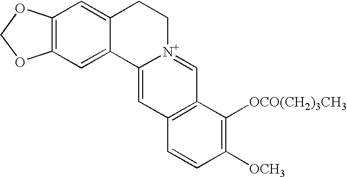 |
| 12-bromoberberine | C₂₀H₁₇BrNO₄ | 415.26 | 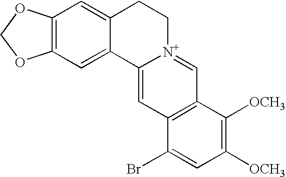 |
| 13-ethoxyberberine | C₂₂H₂₂NO₅ | 380.41 | 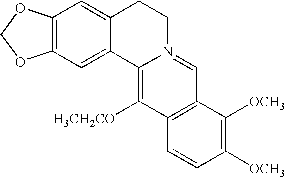 |
| 13-ethylberberine | C₂₂H₂₂NO₄ | 364.41 | 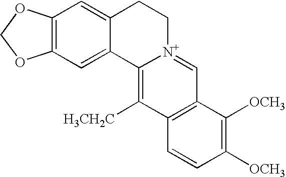 |
| 13-ethylpalmatine | C₂₃H₂₆NO₄ | 380.46 | 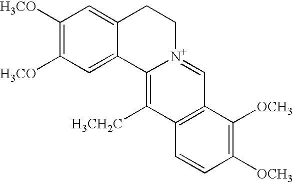 |
| 13-hydroxyberberine | C₂₀H₁₈NO₅ | 352.36 | 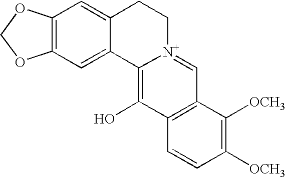 |
| 13-methoxyberberine | C₂₁H₂₀NO₅ | 366.39 | 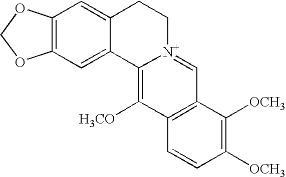 |
| 13-methylberberine | C₂₁H₂₀NO₄ | 350.39 | 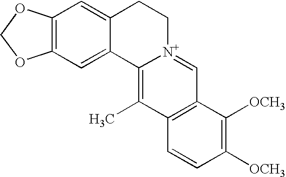 |
| 13-methylberberrubine | C₂₀H₁₈NO₄ | 336.36 | 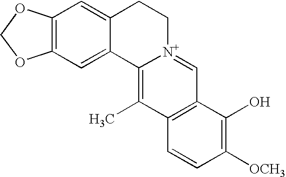 |
| 13-methyldihydroberberine N-methyl salt | C₂₂H₂₄NO₄ | 366.43 | 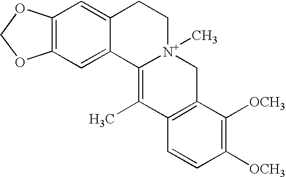 |
| 13-methylpalmatine | C₂₂H₂₄NO₄ | 366.43 | 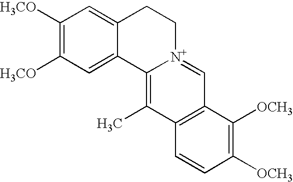 |
| 13-n-butylberberine | C₂₄H₂₆NO₄ | 392.47 | 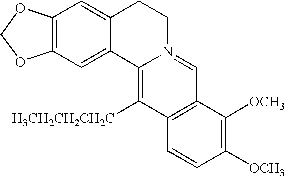 |
| 13-n-butylpalmatine | C₂₅H₃₀NO₄ | 408.51 | 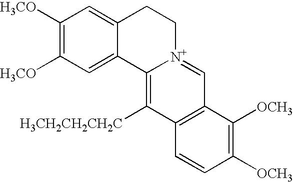 |
| 13-n-hexylberberine | C₂₆H₃₀NO₄ | 420.52 | 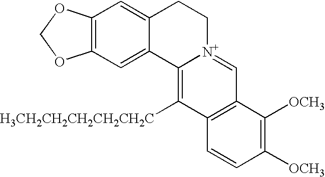 |
| 13-n-hexylpalmatine | C₂₇H₃₄NO₄ | 436.56 | 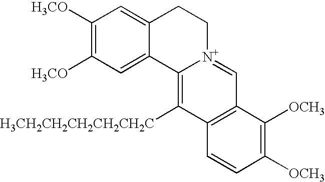 |
| 13-n-propylberberine | C₂₃H₂₄NO₄ | 378.44 | 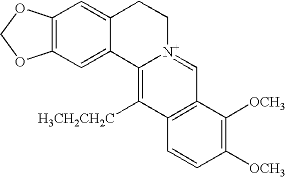 |
| 13-n-propylpalmatine | C₂₄H₂₈NO₄ | 394.48 | 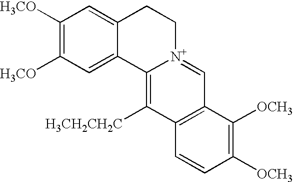 |

Separately, berberine, chlorohexadine, and tetracycline have been used as inhibitors of bacterial growth. However, the present invention is the first disclosure of a combined treatment using berberine and an antibiotic, like chlorohexadine or tetracycline, to treat gingivitis and other diseases and conditions of the mouth, teeth, and gums. The present invention also can be used to treat diseases and conditions directly or indirectly caused by nonoral human pathogens. When berberine is administered in combination with an antibiotic, it has been found that the dose of both the antibiotic and berberine can be reduced by a factor of up to four to achieve the same bacterial growth inhibition observed using either an antibiotic or berberine alone.

The present invention demonstrates that berberine chloride, when administered with CHX or an antibiotic, exerts synergistic growth inhibitory activities. In recent years, oral hygiene products and toothpastes containing goldenseal have become widely available in the U.S. Antibiotics are used in the treatment of periodontal disease. The present invention allows the use of reduced concentrations of CHX and a tetracycline in oral hygiene/therapeutic products to achieve a desired antimicrobial effect, thus minimizing the occurrence of adverse effects attributed to antibiotics and berberine and addressing the problem of pathogen resistance to antibiotics.

The following tests, and tests described in Appendix A, were conducted to illustrate the synergistic potentiating effects achieved by coadministration of berberine and an antibiotic to a mammal, including humans, to treat oral and other human pathogens.

When berberine hydrochloride was administered in combination with chlorhexidine gluconate (CHX) or a tetracycline antibiotic (e.g., tetracycline hydrochloride or doxycycline hydrochloride), a synergistic microbial growth inhibition effect was observed. This active ingredient combination resulted in a lower minimum inhibitory concentration (MIC) compared to the MIC of either agent when measured alone. Five different oral pathogens were evaluated for their susceptibility to combination of these active agents (Streptococcus mutans, Streptococcus gordonii, Porphyromonas gingivalis, Fusobacterium nucleatum, and Anticobacillus actinomycetecomitans). A. actinomycetecomitans was resistant to the test combinations when grown in aerobic conditions, but demonstrated susceptibility under anaerobic conditions.

Berberine chloride, purchased from Sigma (St. Louis, Mo.), was tested to assess whether a different antimicrobial activity existed between a compound isolated from goldenseal and commercially available berberine chloride. The antimicrobial investigations showed no difference in antimicrobial activity between the two sources of barbering chloride.

A combination treatment using berberine hydrochloride and a conventional antimicrobial agent allows for decreased concentrations of both agents in the treatment of mouth, teeth, and gum conditions and diseases, and reduces the occurrence of adverse side effects. This is an important finding because of the increase in resistance to conventional antibacterial treatments. The present invention, therefore, provides a new composition and method for enhanced treatment of mouth, teeth, and gum diseases and conditions, and for the treatment of other diseases and conditions caused, directly or indirectly, by pathogens controlled by berberine and/or an antibiotic.

In addition, two nonoral, human pathogens (Escherichia coli and Staphylococcus aureus) also were evaluated using the combination antibiotic-berberine treatment. The test results showed that when berberine hydrochloride was used in combination with CHX or a tetracycline antibiotic (e.g., tetracycline hydrochloride and doxycycline hydrochloride), a synergistic growth inhibition effect was achieved. These findings show that a composition or method of the present invention can have therapeutic applications in addition to the treatment of oral conditions and diseases.

Berberrubine, structurally similar to berberine, also was evaluated for antimicrobial activity against S. mutans, A. actinomycetecomitans, and P. gingivalis. The test results indicated that berberrubine was not antimicrobial against A. actinomycetecomitans at concentrations less than 300 μml⁻¹. The test results also suggest that berberrubine may be antimicrobial against S. mutans at concentrations greater than 300 μml⁻¹ because, at lower concentrations, growth was hindered, but not inhibited. Berberrubine demonstrated antimicrobial activity against P. gingivalis, at concentrations comparable to berberine (i.e., MIC 17 μg ml⁻¹).

The following table summarizes the approximate amount of an antibiotic, when combined with berberine hydrochloride, required to achieve a predetermined inhibitory activity relative to using the antibiotic alone.

| Species | CHX | Tetracycline (HCL) | Doxycycline (HCL) |
|---|---|---|---|
| S. mutans¹⁾ | <½ | ¼ | ¼ |
| S. gordonii¹⁾ | <½ | <¼ | <¼ |
| P. gingivalis¹⁾ | <½ | ¼ | ¼ |
| F. nucleatum¹⁾ | <½ | ¼ | ¼ |
| E. coli²⁾ | ½ | ½ | ½ |
| S. aureus²⁾ | ½ | ¼ | ⅕ |

¹⁾oral pathogen
²⁾nonoral pathogen

The test results clearly demonstrate that the antimicrobial effects of an antibiotic and berberine on oral and other human pathogens are synergistically potentiated when the agents are coadministered, sequentially or simultaneously. This is an important clinical finding because the amount of antibiotic and berberine required to achieve a desired antimicrobial effect can be substantially reduced, which in turn substantially reduces adverse effects attributed to antibiotic and berberine use.

Berberine combined with an antibiotic, therefore, can be used to potentiate the antimicrobial action of an antibiotic in the treatment of mouth, teeth, and gum diseases, and other diseases and conditions directly or indirectly caused by human pathogens. The test results show that, when combined with berberine administration, antibiotics produce significant antimicrobial control using a much lower dose of antibiotic and berberine, and, therefore, bacterial resistance to the antibiotic and berberine is reduced.

The tests and data show that a combination of an antibiotic and berberine can be administered to mammals in methods of controlling oral and other human pathogens, and treating pathogen-mediated diseases and conditions, especially of the mouth, teeth, and gums. The antibiotic and berberine (i.e., active ingredients) can be formulated in suitable excipients for oral administration, or for parenteral administration. Such excipients are well known in the art. The active agents typically are present in such a composition in an amount of about 0.1% to about 75% by weight, either alone or in combination.

Pharmaceutical compositions containing the active agents, i.e., antibiotic and berberine, of the present invention are suitable for administration to humans or other mammals. Typically, the pharmaceutical compositions are sterile, and contain no toxic, carcinogenic, or mutagenic compounds that cause an adverse reaction when administered. Administration of the pharmaceutical composition can be performed before, during, or after the onset of a disease or condition, e.g., gingivitis, endocarditis, or plaque formation.

The method of the present invention can be accomplished using the active agents as described above individually as the neat compounds, or as a physiologically acceptable salt, solvate, derivative, or prodrug thereof. The active agents can be administered neat, or as a pharmaceutical composition containing either or both entities.

The active agents can be administered by any suitable route, for example, by oral, topical, buccal, sublingual, nasal, percutaneous, i.e., transdermal, or parenteral (including intravenous, intramuscular, subcutaneous, and intracoronary) administration. Parenteral administration can be accomplished using a needle and syringe, or using a high pressure technique, like POWDERJECT™. Typically, the active agents are administered orally, buccally, sublingually, or topically.

The pharmaceutical compositions include those wherein the active ingredients are administered in an effective amount to achieve their intended purpose. More specifically, a “therapeutically effective amount” means an amount effective to prevent development of, to eliminate, or to alleviate a mouth, teeth, gum, or other disease or condition. Determination of a therapeutically effective amount is well within the capability of those skilled in the art, especially in light of the detailed disclosure provided herein.

A “therapeutically effective dose” refers to that amount of the active agents that results in achieving the desired effect. Toxicity and therapeutic efficacy of such active agents can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. A high therapeutic index is preferred. The data obtained from such data can be used in formulating a range of dosage for use in humans. The dosage of the active agents preferably lies within a range of circulating concentrations that include the ED₅₀ with little or no toxicity. The dosage can vary within this range depending upon the dosage form employed, and the route of administration utilized.

The exact formulation, route of administration, and dosage is determined by an individual or physician in view of the individual's condition. Dosage amount and interval can be adjusted individually to provide levels of the active agents that are sufficient to maintain therapeutic or prophylactic effects.

The amount of pharmaceutical composition administered is dependent on the subject being treated, on the subject's weight, the severity of the affliction, the manner of administration, and the judgment of the prescribing physician.

Specifically, for administration to a human in the curative or prophylactic treatment of oral and other human pathogens, dosages of an antibiotic and berberine, individually, generally are about 10 to about 200 mg daily for an average adult patient (70 kg), typically divided into two to three doses per day or administered as a time-release formulation. Thus, for a typical adult patient, individual dosage forms contain about 0.1 to about 200 mg antibiotic and about 0.1 to about 200 mg berberine, in a suitable pharmaceutically acceptable vehicle or carrier, for administration in single or multiple doses, once or several times per day. Dosages for intravenous, buccal, topical, or sublingual administration typically are about 0.1 to about 10 mg/kg per single dose as required. In practice, the physician determines the actual dosing regimen which is most suitable for an individual patient, and the dosage varies with the age, weight, and response of the particular patient. The above dosages are exemplary of the average case, but there can be individual instances in which higher or lower dosages are merited, and such are within the scope of this invention.

The active agents of the present invention can be administered alone, or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the active agents into preparations which can be used pharmaceutically.

These pharmaceutical compositions can be manufactured in a conventional manner, e.g., by conventional mixing, dissolving, granulating, dragee-making, emulsifying, encapsulating, entrapping, or lyophilizing processes. Proper formulation is dependent upon the route of administration chosen. When therapeutically effective amounts of the active agents are administered orally or topically, the composition typically is in the form of a tablet, capsule, powder, solution, or elixir. When administered in tablet form, the composition can additionally contain a solid carrier, such as gelatin or an adjuvant. The tablet, capsule, and powder contain about 5% to about 95% of an active agent of the present invention, and preferably from about 25% to about 90% compound of the present invention. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.5% to about 90% by weight of active agents, and preferably about 1% to about 50% of an active agent.

The active agents can be readily combined with pharmaceutically acceptable carriers well known in the art. Such carriers enable the active agents to be formulated as tablets, pills, dragees, capsules, liquids, gels, syrups, slurries, suspensions, and the like, for oral ingestion by a patient to be treated. Pharmaceutical preparations for oral use can be obtained by adding the active agents with a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The active agents can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of the active agents can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

In addition to the formulations described previously, the active agents also can be formulated as a depot preparation. Such long-acting formulations can be administered by implantation (for example, subcutaneously or intramuscularly) or by intramuscular injection. Thus, for example, the active agents can be formulated with suitable polymeric or hydrophobic materials (for example, as an emulsion in an acceptable oil) or ion exchange resins, or as sparingly soluble derivatives, for example, as a sparingly soluble salt.

In particular, the active agents can be administered orally, buccally, or sublingually in the form of tablets containing excipients, such as starch or lactose, or in capsules or ovules, either alone or in admixture with excipients, or in the form of elixirs of suspensions containing flavoring or coloring agents. Such liquid preparations can be prepared with pharmaceutically acceptable additives, such as suspending agents. A compound also can be injected parenterally, for example, intravenously, intramuscularly, subcutaneously, or intracoronarily. For parenteral administration, the compound is best used in the form of a sterile aqueous solution which can contain other substances, for example, salts, or monosaccharides, such as mannitol or glucose, to make the solution isotonic with blood.

For veterinary use, the active ingredients are administered as a suitably acceptable formulation in accordance with normal veterinary practice. The veterinarian can readily determine the dosing regimen and route of administration that is most appropriate for a particular animal.

As stated above, an antibiotic is widely used in the treatment of several disease conditions of the mouth and gums, including gingivitis. A major problem in the use of an antibiotic in an oral application is their adverse side effects. The problem of bacterial resistance to an antibiotic also exists. It has been discovered that using berberine together with an antibiotic synergistically potentiates the antimicrobial action of the antibiotic, thereby allowing the administration of a reduced amount of antibiotic and berberine to achieve a desired result while addressing bacterial resistance associated with antibiotic and berberine use.

To date, no published literature exists disclosing the synergistic effect observed when berberine is used in combination with a conventional antimicrobial agent, such as CHX, or antibiotics, such as a tetracycline, against oral and other human pathogens. The present invention, therefore, is directed to new drug therapies, such as sustained release systems and new oral hygiene compositions.

In addition, goldenseal is a relatively rare plant and only a small portion of commercially available goldenseal is derived from cultivated crops. The present invention is important with respect to identifying the active components of goldenseal that are formulated into efficacious products, such as oral hygiene products. Berberine derivatives or prodrugs also can be used in accordance with the present invention to overcome availability problems associated with naturally occurring berberine and to reduce the costs associated with extracting berberine from plant sources. The synergistic effect of the combined application of berberine and antibiotic also applies to other human pathogens and, therefore, the combination can be used in the treatment of nonoral human conditions and diseases.

In particular, a present composition and method can be used in the following, nonlimiting practical applications:

(1) over-the-counter oral hygiene products (e.g., mouth rinses and dentrifrices) for the control of plaque and gingivitis using a combination of a berberine and an antibiotic;

(2) the combination of berberine chloride and an antibiotic, like CHX, can be used in other oral products, including dental adhesives for orthodontic brackets to reduce plaque accumulation, dental impression material, chewing gum for caries and gingivitis control, press mints, and varnishes;

(3) a therapeutic treatment or an adjunct for treatment of periodontitis.

Modifications and variations of the invention as hereinbefore set forth can be made without departing from the spirit and scope thereof, and, therefore, only such limitations should be imposed as are indicated by the appended claims.

### Appendix A

### Interactions Between Alkaloids Extracted From Hydrastis Canadensis and Conventional Antimicrobial Agents

Hydrastis canadensis L. (Ranunculaceae), a North American plant known commercially as “goldenseal,” has emerged as one of the top five herbal supplements in the worldwide market (Govindan and Govindan, 2000). Goldenseal is used in various dietary supplements for the purpose of enhancing the immune response of the body. Goldenseal-containing oral hygiene products are sold over the counter (OTC). However, limited information exists with respect to the efficacy of goldenseal components that contribute to purported oral health benefits (McCann et al., 2000).

Berberine is a plant alkaloid having a long history of medicinal use in both Ayurvedic and Chinese medicine. Berberine is present in Hydrastis canadensis (goldenseal), Coptis chinensis (coptis or goldenthread), Berberis aquifolium (Oregon grape), Berberis vulgaris (barberry), and Berberis aristata (tree turmeric). Berberine can be found in the roots, rhizomes, and stem bark of these plants. Berberine extracts and decoctions have demonstrated significant antimicrobial activity against a variety of organisms, including bacteria, viruses, fungi, protozoans, helminths, and chlamydia (Park et al., 2001; Cowan, 1999; Grippa et al., 1999; Park et al., 1999). The amount of berberine in OTC products is about 50 to 200 mg. Currently, the predominant clinical uses for berberine include treatment of bacterial diarrhea, intestinal parasite infections, and ocular trachoma infections (Harry et al., 1988).

Chlorhexidine (CHX) is a bisbiguanide biocide, and compositions containing CHX have been used as topical disinfectants since the middle 1970s. CHX targets the cytoplasmic membrane of bacteria resulting in the loss of structural organization and integrity, and ultimately the coagulation and precipitation of cytoplasmic constituents. In periodontal therapy, CHX has been used in postoperative rinsings, subgingival irrigation, and as an adjunct to mechanical debriment. CHX has a low level of toxicity, both locally and systemically, and shows no permanent retention in the body (International Health Care Foundation, 2001). CHX usually is delivered in rinsing solutions (typically containing 1-2% CHX), and slow-release devices such as varnishes and slab-like sustained delivery devices (Southard and Godowski, 1998). CHX also can be incorporated into chewing gum, toothpastes, and toothbrushes, or as a disinfectant for dentures in patients with candidal infections (International Health Care Foundation, 2001).

Tetracyclines are broad-spectrum antibiotics that can be delivered by many routes of administration, including oral administration and sustained delivery systems. Tetracyclines inhibit protein synthesis by combining with the small (30S) subunit of the ribosome and inhibiting the binding of aminoscyl-tRNA molecules to the ribosomal A site (Prescott et al., 1999). Tetracyclines generally are considered bacteriostatic. The number of reports directed to bacterial resistance to tetracyclines, both in vitro and in vivo, is increasing, however, tetracyclines still are very useful and are routinely used in periodontal treatment. However, if resistant bacteria constitute potential pathogens, the periodontal disease may not be treated, but rather aggravated further (Slots and Rams, 1990).

Tetracyclines concentrate in periodontal pockets at inhibitory levels 2-4 times higher than in the blood. Tetracyclines also strongly bind to root surfaces and can be released in active form over extended time periods. Sublethal concentrations of tetracyclines reduce adherence and coaggregation properties of a number of disease-associated bacteria, and also inhibit the collagenolytic enzymes of mammalian cells enabling periodontal ligament regeneration (Slots and Rams, 1990).

The present invention shows: a) the antimicrobial activity of goldenseal alkaloids against representative oral pathogens and human pathogens, and b) the synergistic efficacy of berberine administration combined with administration of an antibiotic, like CHX, tetracycline (Tet), colistin sulfate (Col), or doxycycline (Dox), against oral and other human pathogens.

Materials and Methods

Extraction of Alkaloids

The alkaloids extracted from goldenseal were berberine, hydrastine, canadaline, canadine and isocorypalmine. A crude extract also was obtained. A berberine derivative, i.e., berberrubine, also was tested, but was not isolated from goldenseal. Berberine and hydrastine were formulated as a chloride salt and a hydrochloride salt, respectively, which eliminated solubility problems associated with the original compounds.

Growth Conditions

Five representative oral pathogens were used as the test organisms, i.e., Streptococcus mutans, Streptococcus gordonii, Porphymonas gingivalis, Fusobacterium nucleatum, and Actinobacillus actinomycetemcomitans. Cultures were obtained from the University of Illinois-Chicago culture collection at the College of Dentistry. S. mutans, S. gordonii, and A. actinomycetemcomitans were grown in Brain Heart Infusion broth (Difco, Becton Dickinson and Co., Sparks, Md., USA). P. gingivalis was grown using defined media containing (1⁻¹):30 g Tryptic Soy Broth (Difco, USA); 5 g Yeast Extract (Difco, USA); 0.5 g 1-cysteine hydrochloride (Sigma, USA); 0.3 μg menadione (Sigma, USA) and 5 μg hemin (Sigma, USA). F. nucleatum was grown in Schaedler Broth (Difco, USA). Three human pathogens also were tested, Escherichia coli, Staphylococcus aureus, and Pseudomonas aeruginosa. Cultures were obtained from the University of Illinois-Chicago culture collection at the College of Medicine. E. coli, St. aureus, and P. aeruginosa were grown in Luria-Bertani (LB) broth (Difco, USA).

Antibacterial Determination

The antimicrobial activity of the alkaloids (as minimum inhibitory concentrations, or MIC) were evaluated using a microdilution assay in 96-well microtiter plates (Fisher Scientific, USA). The tested compounds were berberine (extracted from goldenseal, B-G), berberine chloride (commercial, Sigma, USA, B-S), berberine chloride goldenseal (BCG), canadaline, hydrastine, hydrastine chloride, canadine, isocorypalmine, berberrubine (against S. mutans, A. actinomycetemcomitans, and P. gingivalis only at concentrations of 0-300 μg ml⁻¹), and the crude extract (at concentrations of 0-500 μg ml⁻¹). CHX was used as a positive control at concentrations of 0.02 to 10 μg ml⁻¹. Only berberine (B-G and B-S) was tested against E. coli, St. aureus, and P. aeruginosa.

Overnight cultures of each species were centrifuged (2400×g, 10 minutes), washed twice with 0.05 M phosphate buffered saline (PBS, pH 6.8), and re-suspended. The cell suspension was adjusted using a Cecil Spectrophotometer Series 601 (Milton Roy, Rochester, N.Y.) giving a final concentration in each well of 5×10⁵ colony forming units (CFU) ml⁻¹ for the aerobic species and 5×10⁶. CFU ml⁻¹ for the anaerobic species, in accordance with NCCLS recommendations. All plates were incubated at 37° C. under appropriate atmospheric conditions. S. mutans and S. gordonii were incubated aerobically (also E. coli, S. aureus, and P. aeruginosa). F. nucleatum and P. gingivalus were incubated anaerobically (anaerobic chamber, Ionia Scientific Inc., Marietta, Ohio) in 10% H₂, 5% CO₂, and 85% N₂. A. actinomycetemcomitans was incubated both aerobically and anaerobically. Growth was monitored spectrophotometrically (A₆₆₀ₙₘ) at 0, 24, and 48 h using a Power Wave 200 Microplate Scanning Spectrophotometer (BioTek Instruments, USA). The readings were captured and analyzed using the associated KC4 software, Version 2, Revision 12 (BioTek Instruments, USA). Because berberine and other compounds, for example, berberrubine, are colored, the MIC of the test compounds was defined as the minimum concentration of compound limiting turbidity equal to the compound color blank. The compound controls included inoculated growth medium without test compounds.

Determination of Minimum Bactericide Concentrations (MBC)

The MBC of the compounds against each test species was performed and determined as described in the previous section for MIC determination. After 48 h, 100 μl of culture was removed from wells containing the compound concentrations at or higher than the MIC. Serial dilutions (10⁻¹ to 10⁻⁵) were performed for each sample, and 100 μl of the appropriate dilutions was plated out in triplicate onto appropriate agar. After 48 h, and incubation at 37° C. under appropriate atmospheric conditions, the number of CFU ml⁻¹ were enumerated. The MBC was defined as the lowest concentration of compound that killed 99.9% of the test bacterial population.

Combination Determination

The alkaloids were tested in pair-wise combinations (fractional inhibitory concentrations, FIC) against all the bacteria to determine whether interactions were antagonistic (FIC>1), additive (0.5<FIC<1), or synergistic (FIC<0.5). The FIC was calculated by the following: (MIC A_{combination}/MIC Aₐₗₒₙₑ)+(MIC B_{combination}/MIC Bₐₗₒₙₑ). Where A represents compound 1 and B represents compound 2. The antibacterial efficacy of the alkaloids also were tested in combination with tetracycline HCl (Tet HCl) (0.2 to 25 μg ml⁻¹) (Sigma Chemicals, St. Louis, Mo.), doxycycline HCl (Dox HCl) (0.2 to 25 μg ml⁻¹) (Sigma, USA), and CHX (0.02 to 10 μg ml⁻¹) (Sigma, USA). In the case of A. actinomycetemcomitans and P. aeruginosa, CHX was used at 0.78 to 50 μg ml⁻¹. Berberine (B-CG and B-S) was used at concentrations 0.9 to 250 μg ml⁻¹. The antibacterial efficacy of berberine (1.95 to 500 μg ml⁻¹) was tested in combination against P. aeruginosa with CHX (0.78 to 50 μg ml⁻¹) (Sigma Chemicals) and the polymyxin antibiotic Col (0.04 to 25 μg ml⁻¹) (Sigma Chemicals). The plates were incubated for 24 hours.

Viability Assessment

Two representative organisms, S. mutans and F. nucleatum, were used in this study. Viability of S. mutans to berberine (B-CG) 500 and 1000 μg, ml⁻¹); CHX (20 μg ml⁻¹); B (500 μg ml⁻¹) with CHX (10 μg ml⁻¹); and B (500 μg ml⁻¹) with CHX (5 μg ml⁻¹) was tested. Viability of F. nucleatum to B (500 and 1000 μg ml¹); Dox HCL (5 μg ml⁻¹); B (500 μg ml⁻¹) with Dox (2.5 μg ml⁻¹); and B (250 μg ml⁻¹) with Dox (2.5 μg ml⁻¹) also was tested. The bacteria were prepared as previously described and the optical density was adjusted to give a concentration 1×10⁹ CFU ml⁻¹. Aliquots of 4 ml of agent, prepared in sterile water, were placed into sterile test tubes and 1 ml of adjusted cells was added. The control consisted of 4 ml of PBS and 1 ml of cells. Samples were removed at 0, 15, 30, and 60 minutes, serial dilutions were performed, and the appropriate dilutions plated out. The plates were incubated at 37° C. under appropriate atmospheric conditions for up to 72 hours. Viable colony counts were determined and compared to nontreated control Percentage kill was calculated using the formula:
(CFU_{control}−CFRₜᵣₑₐₜ)/CFU_{control}×100.
Log reduction was also calculated on Log₁₀ transformed data.
Effect of Berberine on Cell Surface Hydrophobicity

Relative cell surface hydrophobicity of, the test bacteria was evaluated using a modified method based on Rosenberg et al. (1980). In brief, the cells were collected by centrifugation (2400×g, 10 minutes), washed twice with phosphate buffered saline (PBS) (0.05 M, pH 6.8), adjusted to give a concentration of 1×10⁸ CFU ml⁻¹, and mixed with equal volumes of the test agents (Table 1). After standing for 10 minutes at room temperature, 500 μl of hexanes (Sigma Chemicals) was added and the reaction mixture vigorously agitated for 1 minute. After separating the organic phase was separated from the aqueous phase, the optical density (O.D. A₅₅₀nm) of the aqueous phase was determined. The relative cell surface hydrophobicity was expressed as a percentage of the absorbance of the organic phase
(O.D._{Initial Aqueous}−O.D._{Extracted Organic})/O.D._{Initial Aqueous}×100 100).

TABLE 1
Concentration of agents used for the hydrophobicity assay

| Agent | Concentration (μg ml⁻¹) |
|---|---|
| Control | 0 |
| Berberine (B) | 500 |
| CHX | 5 |
| Tet | 5 |
| Dox | 5 |

| | | | |
|---|---|---|---|
| B | CHX | 125 | 1.25 |
| B | Tet | 125 | 1.25 |
| B | Dox | 125 | 1.25 |

Accumulation and Efflux of Berberine

An assay based on the ability of S. mutans, S. gordonii, and A. actinomycetemcomitans to grow in the presence of methylene blue (Morita et al., 1998), a substrate for the acrAB efflux system was used to screen for efflux activity. These organisms were used because of their higher susceptibility or resistance to berberine. The bacteria were grown on a series of agar plates containing BHI, and/or berberine (62.5, 125, and 250 μg ml⁻¹) and/or methylene blue (50 μg ml⁻¹), and were incubated for 48 hours at 37° C.

Accumulation of berberine in A. actinomycetemcomitans was evaluated using a fluorimetric assay (based in Giraud et al., 2000). Overnight cells were harvested as previously described, washed twice with 50 mM sodium phosphate buffer (pH 7), resuspended in the same buffer at a concentration of 1×10⁹ CFU ml⁻¹. The cells were equilibrated for 10 minutes at 37° C. Berberine was added at a final concentration of 500 μg ml⁻¹. After addition of berberine, 0.5 ml samples were removed at different time intervals. Five minutes after the addition of berberine, the efflux pump inhibitor carbonyl cyanide m-chlorophenylhydrazone (CCCP) was added to the reaction mixture (final concentration 100 μM). The samples were immediately diluted with 1 ml ice-cold sodium phosphate buffer and then were centrifuged for 5 minutes at 560.0×g. The pellet was washed once with 1 ml ice-cold buffer and resuspended in 1 ml of 0.1 M glycine hydrochloride (pH 3) for at least 15 hours at room temperature. The fluorescence of the supernatant was measured (excitation 355 nm and emission 517 nm) with a spectrofluorimeter (Model 810/814, Photon Technology International, Monmouth Junction, N.J.).

Statistical Analysis

All experiments were repeated at least four times. Statistical, analyses were carried out using Microsoft Excel 1997. Descriptive statistics including the mean, standard deviation, coefficient ((SD/mean)×100) of variation were calculated. The results were analyzed for significance using one-way analysis of variance and the post-hoc Tukey's test (ANOVA, P<0.05; MINITAB® 2002, Version 13.32). Linear regression analysis was performed using Microsoft Excel 1997.

Results

Antibacterial Determination

Inhibitory effects against all the bacteria, except A. actinomycetemcomitans, were observed for berberine (B-CG, B-G, and B-S) and the whole extract (WE) (Table 2). There was no difference in antimicrobial activity between B-CG and the commercially available berberine (B-S). A clear difference in susceptibility to berberine was observed among the test species. P. gingivalis and F. nucleatum exhibited greatest susceptibility to berberine and WE, i.e., eight to sixteen times lower than that of the Streptococcal strains. Hydrastine, the major component (2-6% w/w) of goldenseal, did not inhibit growth of the test bacteria at concentrations≦500 μg ml⁻¹. Canadaline, hydrastine, canadine, and isocorypalmine did not exhibit antimicrobial properties at concentrations≦250 μg ml⁻¹. A. actinomycetemcomitans was resistant to the crude extract and all the test compounds at concentrations≦500 μg ml⁻¹.

Berberrubine, an analog of berberine, did not inhibit the growth of A. actinomycetemcomitans at concentrations≦300 μg ml⁻¹. However, berberrubine may be antimicrobial against S. mutans at concentrations higher than 300 μg ml⁻¹ because at lower concentrations growth was limited but not inhibited. Berberrubine demonstrated antimicrobial activity against P. gingivalis at concentrations comparable to berberine (MIC 17 μg ml⁻¹).

TABLE 2
MIC (ug ml⁻¹) of Goldenseal Alkaloids Against Selected Oral
Pathogens

| | Test Bacteria | | | | |
|---|---|---|---|---|---|
| Sample | Sm | Sg | Aa | Fn | Pg |
| Berberine | 125 | 250 | >500 | 15.625 | 15.625 |
| Hydrastine | >500 | >500 | >500 | >500 | >500 |
| Canadine | >500 | >500 | >500 | >500 | >500 |
| Canadaline | >250 | >250 | >250 | >250 | >250 |
| Isocorypalamine | >125 | >125 | >125 | >125 | >125 |
| Whole Extract (WE) | 250 | 500 | >500 | 62.5 | 62.5 |
| CHX | 1.25 | 5 | 50 | 2.5 | 2.5 |

Sm—S. mutans;
Sg—S. gordonii;
Aa—A. actinomycetemcomitans;
Fn—F. nucleatum, and
Pg—P. gingivalis

This assay system allows concomitant evaluation of the minimum concentration that inhibits biofilm growth. The MIC values obtained for each species also represent the concentration that inhibited biofilm formation. There was no difference in antimicrobial activity between B-G and the other berberine-compounds (B-S and B-CG).

Antimicrobial Activity of Berberine in Combination with CHX or Tetracyclines

Combinations of B-CG with CHX, Tet, Dox, Col yielded lower a MIC than when either agent was used alone (Table 3 and Table 4). Growth of P. aeruginosa was not completely inhibited by exposure to berberine, although growth was impaired at the highest concentration (500 μg ml⁻¹). There was no clear additive or synergistic effect observed by combining berberine with either CHX or Col (Table 3).

TABLE 3
MIC (μg ml⁻¹) of the antimicrobial agents alone

| Species | Berberine | CHX | Tet | Dox | Col |
|---|---|---|---|---|---|
| S. mutans | 125 | 1.25 | 1.56 | 0.78 | ND |
| S. gordonii | 250 | 5 | 0.31 | 0.31 | ND |
| P. gingivalis | 23.4 | 1.25 | 0.08 | 0.08 | ND |
| A. actinomycetemcomitans* | >500 | 50 | 25 | 6.25 | ND |
| F. nucleatum | 15.6 | 2.5 | 0.16 | 0.04 | ND |
| E. coli | 250 | 1.25 | 3.06 | 1.53 | ND |
| S. aureus | 250 | 2.5 | 1.53 | 1.53 | ND |
| P. aeruginosa | >500 | 6.25 | ND | ND | 3.13 |

TABLE 4
MIC (μg ml⁻¹) of the antimicrobial agents in combination

| Species | Berberine | CHX | Tet | Dox | Col |
|---|---|---|---|---|---|
| S. mutans | 62.5 | 0.04 | 0.20 | 0.20 | ND |
| S. gordonii | 62.5 | 0.63 | 0.08 | 0.08 | ND |
| P. gingivalis | 7.81 | 0.08 | 0.01 | 0.01 | ND |
| A. actinomycetemcomitans* | 250 | 50 | 12.5** | 6.25 | ND |
| F. nucleatum | 3.9 | 0.31 | 0.02 | 0.10 | ND |
| E. coli | 125 | 0.63 | 1.53 | 0.77 | ND |
| S. aureus | 62.5 | 0.63 | 0.39 | 0.39 | ND |
| P. aeruginosa | 250** | 3.13** | ND | ND | 1.56 |

MIC values represent the mean of at least three separate replicates
*Grown under anaerobic conditions
ND—not determined
**These values represent borderline additive/synergistic interactions

Because berberine (B-CG and B-S) was the only compound that demonstrated antimicrobial activity, no fractional inhibitory concentrations were obtained for the remaining compounds.

The results show that a synergistic growth inhibition effect was observed (Table 4) when berberine (B-CG and B-S) was used in combination with CHX or tetracycline antibiotics (including tetracycline hydrochloride and doxycycline hydrochloride). Typical results of FIC determination are summarized in Table 5. These combinations resulted in a lower minimum inhibitory concentration (MIC) of either agent when measured alone (Table 6).

TABLE 5
Typical results from the FIC determination. This sample demonstrates the response
of S. mutans to combinations of berberine (B-G) and CHX after 48 h
incubation at 37° C.

| | 1 | 2 | 3 | 4 | 5 | 6 | 7 | 8 | 9 | 10 | 11 | 12 |
|---|---|---|---|---|---|---|---|---|---|---|---|---|
| A | 0.063 | 0.058 | 0.057 | 0.059 | 0.059 | 0.062 | 0.062 | 0.061 | 0.319 | 0.062 | 0.053 | 0.048 |
| B | 0.054 | 0.052 | 0.052 | 0.053 | 0.056 | 0.053 | 0.054 | 0.058 | 0.342 | 0.056 | 0.051 | 0.050 |
| C | 0.051 | 0.052 | 0.051 | 0.053 | 0.054 | 0.062 | 0.195 | 0.266 | 0.340 | 0.136 | 0.051 | 0.051 |
| D | 0.053 | 0.052 | 0.052 | 0.053 | 0.453 | 0.355 | 0.343 | 0.344 | 0.354 | 0.328 | 0.059 | 0.050 |
| E | 0.050 | 0.050 | 0.051 | 0.052 | 0.433 | 0.360 | 0.343 | 0.348 | 0.350 | 0.345 | 0.428 | 0.051 |
| F | 0.049 | 0.053 | 0.050 | 0.052 | 0.433 | 0.349 | 0.358 | 0.351 | 0.346 | 0.338 | 0.351 | 0.051 |
| G | 0.051 | 0.051 | 0.051 | 0.053 | 0.433 | 0.346 | 0.351 | 0.346 | 0.352 | 0.354 | 0.364 | 0.052 |
| H | 0.331 | 0.336 | 0.332 | 0.329 | 0.341 | 0.354 | 0.352 | 0.339 | 0.341 | 0.352 | 0.359 | 0.051 |

CHX was diluted from left to right at 10 to 0.08 μg ml⁻¹ (rows 1 to 8), berberine was diluted from top to bottom at 250 to 0.09 μg ml⁻¹ (columns 1 to 8). Column 9 was the cell control, column 10 was berberine alone (250 to 0.09 μg ml⁻¹); and column 11 was CHX alone (10 to 0.08 μg ml⁻¹). Row H was the solvent control (8% DMSO, H1 to H8). Column 12 was the media blank. The dilution assay was designed such that A1 contained the highest concentration of both agents (berberine 250 μg ml⁻¹ and CHX 10 μg ml⁻¹). The final concentration of berberine in wells A2 to A8 was 125 μg ml⁻¹. The FIC value was calculated from well C6. FIC was calculated by: (MIC A_{combination}/MIC Aₐₗₒₙₑ)+(MIC B_{combination}/MIC Bₐₗₒₙₑ). For C6, A represents berberine and B represents CHX:Berberine (31.25/125)+CHX (0.3125/1.25)=FIC of 0.5, exhibiting a synergistic response.

TABLE 6
Approximate amount of CHX, tetracycline,
or doxycycline required when combined with berberine
(B-CG and B-S) relative to when the agent is used alone

| Species | CHX | Tetracycline-HCl | Doxycycline-HCl |
|---|---|---|---|
| S. mutans | <½ | ¼ | ¼ |
| S. gordonii | <½ | <¼ | <¼ |
| P. gingivalis | <½ | ¼ | ¼ |
| F. nucleatum | <½ | ¼ | ¼ |
| E. coli | ½ | ½ | ½ |
| S. aureus | ½ | ¼ | ½ |

Viability Assessment

Viability of both S. mutans and F. nucleatum was reduced by exposure to B-CG and was dependent on concentration and exposure time. This was most apparent for F. nucleatum (Table 7). Viability of F. nucleatum continued to decline during exposure to B-CG throughout the experimental period. In comparison, B-CG was most active during the first 15 minutes of exposure against S. mutans. The activity of B-CG was comparable to CHX against S. mutans and F. nucleatum (Table 7, P>0.05).

TABLE 7
Bactericidal activity of berberine against S. mutans and F. nucleatum

Viability after 60 min
exposure (mean Log₁₀ reduction of CFU)

| Species | B-CG (500) | B-CG (1000) | CHX (20) |
|---|---|---|---|
| S. mutans | 0 | 1* | 1.69* |
| F. nucleatum | 2.77* | 3.22* | 3.09* |

*Indicates a significant reduction compared to the control at a 95% confidence interval.

Combining B-CG and CHX yielded a reduction in viability that was comparable to viability reduction when the agents were used alone at higher concentrations against S. mutans as shown in Table 8 and Table 9 (P<0.05).

TABLE 8
Bactericidal activity of combinations of B-CG and CHX against
S. mutans

| | Viability after 60 min exposure | | | | |
|---|---|---|---|---|---|
| | (mean Log₁₀ reduction of CFU) | | | | |
| | B-CG | B-CG | CHX | B-CG (500) | B-CG (500) |
| Species | (500) | (1000) | (20) | & CHX (10) | & CHX (5) |
| S. mutans | 0 | 1 | 1.69 | 1.18 | 0.22 |

TABLE 9
Comparison of mean Log₁₀ reduction in viability of S. mutans after 60
min exposure evaluated using Tukey's post-hoc comparison test

| | | | | B-CG (500) |
|---|---|---|---|---|
| | B-CG (500) | B-CG (1000) | CHX (20) | & CHX (10) |
| B-CG (1000) | NSD | | | |
| CHX (20) | SD | NSD | | |
| B-CG (500) & | NSD | NSD | NSD | |
| CHX (10) | | | | |
| B-CG (500) & | NSD | NSD | SD | NSD |
| CHX (5) | | | | |

SD indicates a significant difference in the reduction of viability between treatments at a 95% confidence interval.
NSD indicates no significant difference in the reduction of viability between treatments at a 95% confidence interval.

Combining B-CG and Dox yielded a reduction in viability that was comparable to viability reduction when the agents were used alone at higher concentrations against F. nucleatum as shown in Table 10 and Table 11 (P<0.05). The concentration required to achieve comparable reduction in viability when the agents were used in combination was reduced one-half for Dox (5 μg ml⁻¹) and four times for berberine (250 μg ml⁻¹) (Table 10 and Table 11).

TABLE 10
Bactericidal activity of combinations of B-CG with Dox against
F. nucleatum

| | Viability after 60 min exposures | | | | |
|---|---|---|---|---|---|
| | (mean Log₁₀ reduction of CFU) | | | | |
| | B-CG | B-CG | Dox | B-CG (250) | B-CG (250) |
| Species | (500) | (1000) | (10) | & Dox (5) | & Dox (2.5) |
| F. nucleatum | 2.77 | 3.22 | 1.56 | 3.37 | 2.09 |

TABLE 11
Comparison of mean Log₁₀ reduction in viability of F. nucleatum after 60
min exposure evaluated using Tukey's post-hoc comparison test

| | | | | B-CG (500) |
|---|---|---|---|---|
| | B-CG (500) | B-CG (1000) | Dox (10) | & Dox (5) |
| B-CG (1000) | NSD | | | |
| Dox (10) | NSD | NSD | | |
| B-CG (250) & | NSD | NSD | SD | |
| D9x (5) | | | | |
| B-CG (250) & | NSD | NSD | NSD | NSD |
| Dox (2.5) | | | | |

SD indicates a significant difference in the reduction of viability between treatments at a 95% confidence interval.
NSD indicates no significant difference in the reduction of viability between treatments at a 95% confidence interval.

Effect of Berberine on Cell Surface Hydrophobicity

The reduction of relative surface hydrophobicity is outlined in Table 12. B-CG was shown to reduce hydrophobicity between 15% and 30%. CHX, Tet, and Dox did not appear to affect the relative hydrophobicity of the cells. Reduction in cell surface hydrophobicity still was observed, however, when the agents were tested in combination.

The assay demonstrated that A. actinobacillus was less hydrophobic compared to the other bacteria. When A. actinobacillus was exposed to the agents in combination and alone, an increase surface hydrophobicity of about 25% was observed.

TABLE 12
Typical reduction scores (%) on relative cell surface hydrophobicity

| Treatment | S. mutans | S. gordonii | F. nucleatum | A. actinomycetemcomitans | P. gingivalis |
|---|---|---|---|---|---|
| B-500 | 14.8 | 26.1 | 29.9 | −31.6 | 26.7 |
| Tet-5 | 4.3 | 9.7 | 0.4 | −21.55 | 0.7 |
| Dox-5 | 8.1 | 7.7 | −3.5 | −4.1 | 1.7 |
| CHX-5 | 5.1 | −6.3 | 3.7 | −2.6 | −2.7 |
| B-CHX | 7.2 | 19.8 | 26.1 | −26.1 | 14.3 |
| (125 & | | | | | |
| 1.25) | | | | | |
| B-Tet | 9.4 | 18.6 | 28.2 | −24.7 | 16.4 |
| (125 & | | | | | |
| 1.25) | | | | | |
| B-Dox | 9.4 | 23.4 | 28.9 | −24.8 | 23.5 |
| (125 & | | | | | |
| 1.25) | | | | | |

Accumulation and Efflux of Berberine

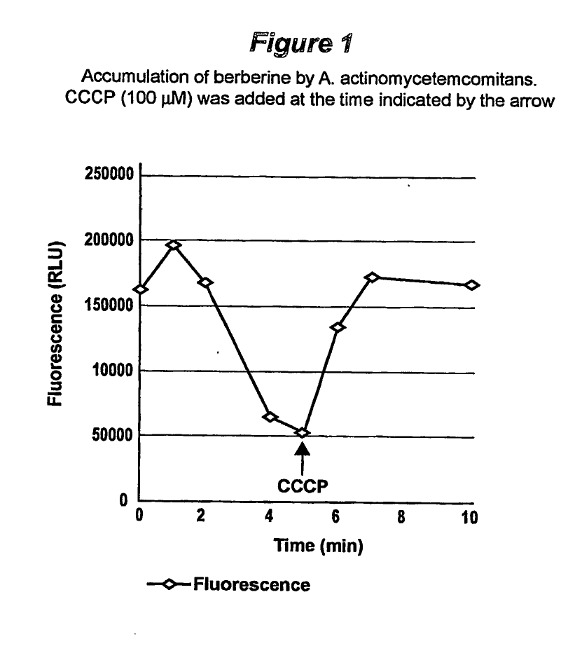

S. mutans and S. gordonii did not grow in the presence of methylene blue (MB) (50 μg/ml) and only grew in the presence of B-CG at 62.5 μg/ml. A. actinomycetemcomitans grew in the presence of B-CG at all concentrations at MB. When efflux of berberine by A. actinomycetemcomitans was monitored spectrofluorometrically an initial accumulation was observed followed by a decline (FIG. 1). Addition of CCCP induced a very rapid and marked increase in berberine accumulation (FIG. 1).

Discussion

To date there is no published reference disclosing the enhanced effect observed when berberine is used in combination with a conventional antimicrobial agents, such as CHX, or antibiotics, such as tetracyclines, against oral and human pathogens.

With the increase in the number of reports of bacterial resistance to conventional treatment, attention now is turning to the management of infections with nonconventional antimicrobials (Kristiansen et al., 1997; Appleman et al., 2000).

Plants remain a major source of medicinal compounds. It is estimated that 20,000 plant species are used for medicinal purposes (Penos, 1983). Medicinal plants have been used throughout history, and efficacy often is associated with anecdotal folklore. Further estimations suggest that 74% of 119 plant derived drugs were discovered as a result of chemical studies to isolate the active components responsible for their traditional use (Farnsworth et al., 1985). At least 25% of recent medicinal prescriptions contain one active-compound from plant species (Haq, 2000), and the estimated value of these prescriptions containing plant-derived compounds is worth about $10 billion dollars in the U.S. alone (Duke, 1990).

The antimicrobial activity of berberine has been reported. Berberine, isolated from the Chinese medicinal plant, Coptidis rhizoma, demonstrated antimicrobial activity against seven periodontal pathogens (Hu et al., 2000). The investigators also proposed that a clinical application for berberine in the treatment of periodontal diseases might exist. Scazzocchio et al., (2001) examined the antibacterial activity of β-hydrastine, canadine, and canadaline, isolated from H. canadensis, against S. aureus, E. coli, P. aeruginosa, and S. sanguis. They reported β-hydrastine was inactive against all the tested organisms, but that canadaline and canadine were antimicrobial at comparable concentrations to berberine. The reduced antimicrobial efficacy of the crude extract against the test organisms therefore may be attributed to the presence of these other alkaloids and other components.

H. canadensis is considered a rare plant. The present invention highlights the importance of identifying the major antimicrobial contributions of alkaloids from plants, and illustrates that use of extract components in combination with standard antibiotics provides a synergistic therapeutic effect.

The preferred growth mode for many species of bacteria is biofilm. Biofilm formation takes place when bacteria attach to a surface from a liquid phase, producing exopolymeric substances that aid in the continued attachment to the surface and offer protection from the external environment. Bacteria in the form of biofilms have been reported to be up to 1000 times more resistant to treatment than the bacteria present in liquid phase (Lewis, 2001; Costerton et al., 1999). This is a serious problem for the efficacious treatment of infections (Costerton et al., 1999). Harry et al., (1988) reported that berberine sulfate at sub-MIC levels inhibited the adherence of S. pyogenes to epithelial cells. The results of these tests have shown that berberine and combinations thereof were effective at inhibiting biofilm formation of all the test species except A. actinomycetemcomitans.

Because the conventional antimicrobial agents CHX, Tet, and Dox did not affect surface hydrophobicity, beneficial effects-could be achieved by combining these antimicrobial agents with berberine. The results from this investigation indicated that berberine was most likely the main contributing agent in reducing cell hydrophobicity. Cell surface hydrophobicity is an important factor in bacterial attachment to a surface and subsequent biofilm formation (Merritt et al., 2000). It also has been proposed that the hydrophobic effect may be the primary driving force for the adhesion of most pathogens (Oliviera et al., 2001). Oral pathogens that have lost their surface hydrophobicity demonstrated significantly less colonization than the parent strains (Matsumoto et al., 1999). Reducing the attachment capability of oral bacteria and other pathogens would be highly desirable, particularly in those persons with recalcitrant infections.

A. actinomycetemcomitans, a facultative anaerobe and potent periodontal pathogen is known for its resistance to antimicrobial treatment, particularly tetracycline antibiotics (Fives-Taylor et al., 2000). Reversal of resistance by synergy produced as a result of the combination of conventional antibiotics and nonantibiotics has been achieved (Kristiansen et al., 1997). Although the results from this investigation have shown that A. actinomycetemcomitans was not susceptible to this antimicrobial regime, it may be possible to achieve an antimicrobial response if the concentration of agents was increased.

Preliminary investigations have shown that when A. actinomycetemcomitans was incubated under anaerobic conditions, it was more susceptible to treatment, demonstrating similar susceptibility profiles as P. aeruginosa. The results from this investigation demonstrated that an active efflux is involved in A. actinomycetemcomitans resistance to berberine. In addition, the increase in hydrophobicity observed when A. actinomycetemcomitans was exposed to the antimicrobial agents demonstrates this organism's wide-ranging ability to resist antimicrobial treatment and highlights the need for efficacious treatment.

P. aeruginosa increasingly is associated with nosocomial infections both in the U.S. and Europe, particularly pneumonia, and can be fatal for immunocompromised persons. Pseudomonas infections can be spread within hospitals by healthcare workers, medical equipment, sinks, disinfectant solutions, and food. The bacterium also causes serious, problems in individuals suffering from cystic fibrosis, a fatal genetic disorder.

Antibiotic resistance in Pseudomonas infections is prevalent. The lack of novel agents in development calls for a need to reexamine the role of colistin therapy in persons with cystic fibrosis (Beringer, 2001). Colistin is a cationic antibiotic belonging to the polymyxin group. Colistin initially was introduced in the early 1960s, but became redundant by the early 1970s due to reports of its severe toxicity. In this present study, this strain of P. aeruginosa was resistant to berberine at the test concentrations, although impairment of growth was observed when combined with CHX and Col. As with the other antibiotics, Tet and Dox, adverse side effects associated with Col can be reduced when combined with berberine. Other strains of P. aeruginosa may demonstrate greater susceptibility and, therefore, the beneficial effects of combining Col with berberine cannot be disregarded.

CHX is a widely used biocide in antiseptic and disinfectant products, particularly in hospitals. It has broad spectrum efficacy and is much less irritating to tissue than other products (McDonnell et al., 1999). Chlorhexidine is bactericidal and fungicidal to a wide variety of organisms and it is possible that by combining CHX with berberine, that its activity spectrum may be increased. Bacterial resistance to CHX has been reported, specifically S. aureus and S. epidermidis, and there is increasing concern regarding the link between biocide exposure and antibiotic resistance (Russell et al., 2000; Russell, 2000; McDonnell et al., 1999; Russell et al., 1996). Combinations of CHX with a natural antimicrobial agent, such as berberine, may result in a more environmentally acceptable product and may serve to increase the longevity of use and efficacious treatment of oral infections and disinfectant procedures.

Because berberrubine is structurally related to berberine, the results from this investigation suggest that analogs of berberine are antimicrobially active. Synthesized derivatives of protoberberines have been tested against several Candida species (Park et al., 2001). The authors also proposed that semisynthetic protoberberines may have considerable potential as novel antifungal agents that lack host toxicity.

A report by Stermitz et al. (1999) hypothesized that plant species containing berberine may evolve multidrug resistance (MDR) efflux pump inhibitors to potentiate berberine activity against pathogens. Resistance to CHX and tetracycline antibiotics theoretically is related to the presence of MDR efflux pumps in bacterial cell membranes which actively remove the agent from the cell. Protoberberine is an amphiphilic cation, similar to CHX, and demonstrates antimicrobial activities, but also is susceptible to extrusion from bacterial cells by MDR pumps (Stermitz et al., 1999). E. coli has seven different known MDR pump systems that can export structurally unrelated antibiotics (Sulavik et al., 2001). MDR systems have also been identified in S. aureus (Markham et al., 1999), P. aeruginosa (Li et al., 1995), and A. actinomycetemcomitans (Fives-Taylor et al., 2000).

It is theorized, but not relied upon herein, that the observed synergistic/additive response is related to the inhibition of MDR systems present in the cell membrane. It also is theorized that when berberine is used in combination with an antibiotic, cells are saturated and are rendered incapable of removing the agents simultaneously.

In addition, MDR pump systems are usually ATP dependent. The increased susceptibility of the anaerobic species to berberine and combinations thereof may be related to the reduced amounts of ATP produced under anaerobic conditions (Prescot et al., 1999).

It is further proposed that when berberine is used in combination with other antimicrobial agents, which are substrates for MDR systems, that the same additive/synergistic effect could be achieved. It is possible to achieve the same effect with non-MDR substrates, because many bacteria possess different MDR systems.

A report by Sheng et al. (1997) reported that when berberine was used in combination with pyrimethamine, good results for treating patients with chloroquine-resistant malaria was observed. Another plant alkaloid, reserpine, has been shown to inhibit the efflux of the fluoroquinolone antibiotic norfloxacin in wild type S. aureus by at least four-fold (Markham et al., 1999). However, the authors also proposed that reserpine could not be used to potentiate the activities of fluoroquinolones because of neurotoxicity to humans at the concentrations required for efflux inhibition.

If administered in sufficiently high doses, a goldenseal alkaloid can produce toxic symptoms ranging from mouth irritation, nausea, vomiting, and diarrhea to cardiac depression and central nervous system paralysis (Tice, 1997). It also has been reported that berberine (100 mg) reduced the efficacy of tetracycline in people with cholera by reducing tetracycline absorption (Khin-Maung et al., 1985). However, in another report the findings were inconclusive as to whether berberine significantly interacted tetracycline antibiotics (Raddani et al., 1987).

At present, CHX is the “gold standard” of all plaque control agents and is included in oral hygiene products. However, CHX is a prescription drug in the US. In addition, CHX has many side effects, including undesirable taste, tooth discoloration, and increased calculus (tartar) formation in users, although no permanent retention in the body is observed (international Health Care Foundation, 2001). Tetracyclines are conventional antibiotics and are routinely used in the treatment of periodontal disease because of their unique ability to accumulate in the pocket area. However, like many conventional antibiotics, many bacteria, especially important human pathogens, have developed a resistance to tetracyclines. Tetracyclines also have many side effects including, gastrointestinal discomfort, nausea, vomiting, diarrhea, and permanent tooth discoloration in children up to 8 years old.

Berberine is found in many health supplements in amounts exceeding 50 mg. Antibiotics used in periodontal treatment include tetracycline and doxycycline, and are administered typically at 100 mg (doxycycline) to 500 mg (tetracycline) with concentrations in the gingival crevicular fluid ranging from 1 to 8 μg ml⁻¹ (doxycycline) and 2 to 12 μg ml⁻¹ (tetracycline) (Slots and Rams, 1990). The present invention shows that when berberine is administered in combination with an antibiotic, like CHX, tetracycline, or doxycycline, approximately half to one quarter the amount of a berberine and an antibiotic is required to achieve the desired growth inhibition compared to when either agent is used alone. Synergy observed in vitro also is evident in vivo, and can lead to the development of drug combinations for the management of infections that are difficult to treat with one antibiotic alone (Kristiansen and Amaral, 1997). If a combination treatment using berberine and a conventional antimicrobial agent is employed, decreased concentrations of both agents can be used, thereby reducing the occurrence and/or severity of adverse side effects.

Dental plaque frequently is associated with oral diseases including dental caries and periodontal (gum) disease. Plaque control is of utmost importance for the prevention of these diseases. Plaque control can be achieved by mechanical (e.g., brushing or flossing) and chemical means. For the latter, antimicrobial agents have been incorporated, into oral hygiene products, such as toothpastes and mouth rinses, to control plaque and gingivitis/periodontal disease. Therefore, a composition containing reduced levels of CHX, tetracycline, doxycycline, or other antibiotics, that minimizes occurrence or severity of the above-mentioned adverse effects while achieving the desired antimicrobial effect would be a significant advance in the art.

The efficacy of the combination treatment against two well known human pathogens, E. coli and S. aureus, also was demonstrated in vitro. The results indicate that when berberine chloride is used in combination with CHX or a tetracycline antibiotic (including tetracycline hydrochloride and doxycycline hydrochloride), a synergistic growth inhibition effect is observed. These findings suggest that the present combination treatment can provide new therapeutic agents for treatment of oral and other diseases.

In summary, the above results demonstrate potentiative interactions between an antibiotic and a berberine in a novel management of oral and nonoral infections.

### REFERENCES

P. Beringer, Current Opinion Polmonary Medicine, 7:434-440 (2001).
J. W. Costerton et al., Science, 284:1318-1322 (−1999).
M. M. Cowan, Clinical Microbiology Reviews, 12:564-582 (1999).
J. A. Duke, Promising phytochemicals. In: Advances in New Crops. Ed. J. Janik and J. E. Simon. Portland, Timber Press. pp 491-498 (1990).
N. R. Farnsworth et al., Medicinal plants in therapy. VMO Bull. (1985).
D. H. Fine et al., International Journal of Antimicrobial Agents, 9:235-238 (1998).
P. M. Fives-Taylor et al., Periodontology, 20: 36-67 (2000).
E. Giraud et al., Antimicrobial Agents and Chemotherapy, 44:1223-1228 (2000).
M. Govindan et al., Fitoterapia, 3:232-235 (2000).
E. Grippa et al., Bioscience Biotechnology and Biochemistry, 9:1557-1562 (1999)
N. Haq, In vitro productions of bioactive compounds from medicinal and aromatio plants. TelMedPak Agriculture website (2000).
D. S. Harry et al., Antimicrobial Agents and Chemotherapy, 32:1370-1374 (1988).
J. P. Hu et al., Oral Diseases, 6:297-302 (2000).
International Health Care Foundation, in collaboration with Departments of Cardiology and Periodontology, Sweden. Internet source.www.ihcf.li/publi/chxrew.html (2001).
Khin-Maung-U et al., British Medical Journal, 6509:1601-5 (1985).
J. E. Kristiansen et al., Journal of Antimicrobial. Chemotherapy, 40: 319-327 (1997).
R. J. W. Lambert et al., Journal of Applied Microbiology, 91: 453-462 (2001).
K. Lewis, Antimicrobial Agents and Chemotherapy, 45:999-1007 (2001).
P. N. Markham et al., Antimicrobial Agents and Chemotherapy, 43:2404-2408 (1999).
M. Matsumoto et al., Caries Research, 33:441-445 (1999).
G. McDonnell et al., Clinical Microbiology Reviews, 12:14-179 (1999).
K. Merritt et al., in Handbook of Bacterial Adhesion: Principles, Methods, and Applications (Ed. Y. H. An et al), Humana Press, Totowa, N.J., USA, pp. 53-72 (2000).
Y. Morita et al., Antimicrobial Agents and Chemotherapy, 42:1778-1782 (1998).
R. Oliviera et al., in Bioflim Community Interactions: Chance or Necessity? (Ed. P. Gilbert et al.), pp. 11-22 (2001).
K. S. Park et al., Journal of Antimicrobial Chemotherapy, 5:667-674 (1999).
K. S. Park et al., Journal of Antimicrobial Chemotherapy, 47:513-519 (2001).
G. Penos, Index Plantarum Medicinalium Totis Mundi Eorumque Synymorum (EMPLED). Organisation of Educational Medicine (1983).
L. M. Prescot et al., Microbiology. 4th Ed. McGraw Hill, Boston (1999).
G. H. Rabbani et al., Journal of Infectious Diseases, 5:979-84 (1987).
A. D. Russell et al., Microbios, 85:45-65 (1996).
A. D. Russell et al., Journal Pharmacy and Pharmacology, 52:227-233 (2000).
A. D. Russell et al., Journal of Hospital Infection, 44:1-3 (2000).
F. Scazzocchio et al., Planta Med., 67:561-564 (2001).
W. D. Sheng et al., East African Medical Journal, 5:283-284 (1997).
J. Slots et al., Journal of Clinical Periodontology, 17:479-493 (1990).
G. L. Southard et al., International Journal of Antimicrobial Agents, 9:239-253 (1998).
F. R. Stermitz et al., Journal of Natural Products, 63: 146-149 (1999).
D. J. Stickler et al., British Journal of Clinical Practice, 25 (Suppl): 23-28 (1983).
M. C. Sulavik et al., Antimicrobial Agents and Chemotherapy, 45:1126-1136 (2001).
R. Tice, R. Goldenseal (Hydrastis canadensis L.) and two of its constituent alkaloids, berberine [2086-83-1] and hydrastine[118-08-1]. Review of Toxicological Literature. Internet source (1997).
www.server.niehs.nih.gov/htdocs/Chem_Backg round/ExecSumm/GoldenSeal.html
http://www.sbwise.com/ingredients/goldense al.html#SAFETY FACTORS & TOXICITY

What is claimed is:

1. A method of controlling oral pathogens comprising administering to a mammal in need thereof a therapeutically effective amount of an antimicrobial or an antibiotic, and a therapeutically effective amount of a berberine.

2. The method of claim 1 wherein the antimicrobial or antibiotic and berberine are administered simultaneously.

3. The method of claim 2 wherein the antimicrobial or antibiotic and berberine are administered from a single composition.

4. The method of claim 2 wherein the antimicrobial or antibiotic and berberine are administered from separate compositions.

5. The method of claim 1 wherein the antimicrobial or antibiotic and berberine are administered sequentially.

6. The method of claim 5 wherein the antimicrobial or antibiotic is administered prior to the berberine.

7. The method of claim 5 wherein the berberine is administered prior to the antimicrobial or antibiotic.

8. The method of claim 1 wherein the antimicrobial or antibiotic is selected from the group consisting of a chlorhexidine, a tetracycline, and a mixture thereof.

9. The method of claim 1 wherein the antimicrobial or antibiotic is selected from the group consisting of chlorhexidine, chlorhexidine dihydrochloride, chlorhexidine diacetate salt hydrate, chlorhexidine digluconate, alexidine, alexidine dihydrochloride, tetracycline, tetracycline hydrochloride, doxycycline, doxycycline hydrochloride, and mixtures thereof.

10. The method of claim 1 wherein the antimicrobial or antibiotic comprises chlorhexidine, tetracycline, doxycycline, or a salt thereof.

11. The method of claim 1 wherein the berberine is selected from the group consisting of free berberine, berberine sulfate, berberine bisulfate, berberine hemisulfate, berberine chloride, berberrubine, coptisine, palmatine, 8-ethyl-12-bromoberberine, 8-ethylberberine, 8-methoxyberberine, 8-methylberberine, 8-n-butyl-12-bromoberberine, 8-n-butylberberine, 8-n-hexyl-12-bromoberberine, 8-n-propyl-12-bromoberberine, 8-n-propylberberine, 8-phenyl-12-bromoberberine, 8-phenylberberine, 9-O-acetylberberrubine, 9-O-benzoylberberrubine, 9-O-ethyl-13-ethylberberrubine, 9-O-ethylberberrubine, 9-O-lauroylberberrubine, 9-O-valerylberberrubine, 12-bromoberberine, 13-ethoxyberberine, 13-ethyl-berberine, 13-ethylpalmatine, 13-hydroxyberberine, 13-methoxyberberine, 13-methylberberine, 13-methylberberubine, 13-methyldihydroberberine N-methyl salt, 13-methylpalmatine, 13-n-butylberberine, 13-n-butylpalmatine, 13-n-hexylberberine, 13-n-hexyl-palmatine, 13-n-propylberberine, 13-n-propyl-palmatine, and mixtures thereof.

12. The method of claim 1 wherein the berberine comprises berberine chloride, berberine sulfate, or a mixture thereof.

13. The method of claim 1 wherein the antimicrobial or antibiotic is administered at a dose lower than a dose of the same antibiotic administered alone to achieve a predetermined control of oral pathogens.

14. The method of claim 13 wherein lower dose is about one-half of the antimicrobial or antibiotic dose when used alone.

15. The method of claim 13 wherein lower dose is about one-quarter of the antimicrobial or antibiotic dose when used alone.

16. The method of claim 1 wherein the berberine is administered at a dose lower than a dose of the same berberine administered alone to achieve a predetermined control of oral pathogens.

17. The method of claim 16 wherein lower doses in about one-half of the berberine dose when used alone.

18. The method of claim 16 wherein lower dose is about one-quarter of the antimicrobial or antibiotic dose when used alone.

19. The method of claim 1 wherein the mammal is a human.

20. A method of treating a mouth, tooth, or gum disease or condition comprising administering to a mammal in need thereof a therapeutically effective amount of an antimicrobial or an antibiotic, and a therapeutically effective amount of a berberine.

21. The method of claim 20 wherein the disease or condition is selected from the group consisting of dental caries, a periodontal disease, gingivitis, periodontitis, endocarditis, a candidial infection, an infection of oral hard or soft tissue, and plaque formation.

22. A method of controlling human pathogens comprising administering to a mammal in need thereof a therapeutically effective amount of an antimicrobial or antibiotic, and a therapeutically effective amount of a berberine.

23. A method of treating a disease or condition caused by a human pathogen comprising administering to a mammal in need thereof a therapeutically effective amount of an antimicrobial or an antibiotic, and a therapeutically effective amount of a berberine.

24. A composition comprising (a) an antimicrobial or an antibiotic, (b) a berberine, and (c) an optional excipient.

25. The composition of claim 24 wherein the antimicrobial or antibiotic is selected from the group consisting of chlorhexidine, chlorhexidine dihydrochloride, chlorhexidine diacetate salt hydrate, chlorhexidine digluconate, alexidine, alexidine dihydrochloride, tetracycline, tetracycline hydrochloride, doxycycline, doxycycline hydrochloride, and mixtures thereof.

26. The composition of claim 24 wherein the berberine comprising free berberine, berberine sulfate, berberine bisulfate, berberine hemisulfate, berberine chloride, berberrubine, coptisine, palmatine, 8-ethyl-12-bromoberberine, 8-ethylberberine, 8-methoxyberberine, 8-methylberberine, 8-n-butyl-12-bromoberberine, 8-n-butylberberine, 8-n-hexyl-12-bromoberberine, 8-n-propyl-12-bromoberberine, 8-n-propylberberine, 8-phenyl-12-bromoberberine, 8-phenylberberine, 9-O-acetylberberrubine, 9-O-benzo-ylberberrubine, 9-O-ethyl-13-ethylberberrubine, 9-O-ethylberberrubine, 9-O-lauroylberberrubine, 9-O-valerylberbertubine, 12-bromoberberine, 13-ethoxy-berberine, 13-ethylberberine, 13-ethylpalmatine, 13-hydroxyberberine, 13-methoxyberberine, 13-methyl-berberine, 13-methylberberubine, 13-methyldihydro-berberine N-methyl salt, 13-methylpalmatine, 13-n-butylberberine, 13-n-butylpalmatine, 13-n-hexyl-berberine, 13-n-hexylpalmatine, 13-n-propylberberine, 13-n-propylpalmatine, and mixtures thereof.

27. An article of manufacture comprising:
(a) a packaged composition comprising an antimicrobial or antibiotic;
(b) a packaged composition comprising a berberine;
(c) an insert providing instructions for a simultaneous or sequential administration of (a) and (b) to control oral pathogens in a mammal; and
(d) a container for (a), (b), and (c).

28. An article of manufacture comprising:
(a) a packaged composition comprising an antimicrobial or antibiotic and a berberine;
(b) an insert providing instructions for administration of (a) to control oral pathogens in a mammal; and
(c) a container for (a) and (b).

29. The article of claim 28 selected from the group consisting of a toothpaste, a mouth rinse, an oral hygiene product, a pharmaceutical product, a chewing gum, a toothbrush, a denture disinfectant, a dentifrice, a dental adhesive, and a dental impression material.
